SEQUENCE LISTING (13 sequences)

SEQ ID NO: 1 - protein, 13 aa, Artificial Sequence
  BSEC-1 peptide structure
TEEISEVNLDAEF

SEQ ID NO: 2 - protein, 13 aa, Artificial Sequence
  BSEC-3 polypeptide structure
TEEISEVNLDAEF

SEQ ID NO: 3 - DNA, 20 bp, Artificial Sequence
  ALF-Capture polypeptide
taaataccattaaaaatgca

SEQ ID NO: 4 - DNA, 20 bp, Artificial Sequence
  ALF-Target polypeptide
tgcatttttaatggtattta

SEQ ID NO: 5 - DNA, 20 bp, Artificial Sequence
  DNA-QTL (20-mer) polypeptide
tgcatttttaatggtattta

SEQ ID NO: 6 - DNA, 17 bp, Artificial Sequence
  DNA-QTL (17-mer)
atttttaatggtattta

SEQ ID NO: 7 - DNA, 20 bp, Artificial Sequence
  ALF Target polypeptide
tgcatttttaatggtattta

SEQ ID NO: 8 - DNA, 20 bp, Artificial Sequence
  G-T Mismatch polypeptide
tgcatttttgatggtattta

SEQ ID NO: 9 - DNA, 20 bp, Artificial Sequence
  T-T Mismatch polypeptide
tgcattttttatggtattta

SEQ ID NO: 10 - DNA, 20 bp, Artificial Sequence
  C-T Mismatch polypeptide
tgcatttttcatggtattta

SEQ ID NO: 11 - DNA, 20 bp, Artificial Sequence
  Double Mismatch polypeptide
tgcatatttaatggaattta

SEQ ID NO: 12 - DNA, 17 bp, Artificial Sequence
  DNA-QTL polypeptide
atttttaatggtattta

SEQ ID NO: 13 - DNA, 15 bp, Artificial Sequence
  PNA-Capture polypeptide
taaataccattaaaa

(19) United States
(12) Patent Application Publication
(10) Pub. No.: US 2004/0175768 A1
(43) Pub. Date: Sep. 9, 2004

(54) Methods of biosensing using fluorescent polymers and quencher-tether-ligand bioconjugates

(76) Inventors: Stuart A. Kushon, Santa Fe, NM (US); Sriram Kumaraswamy, Santa Fe, NM (US); Wensheng Xia, Albuquerque, NM (US); Robert M. Jones, Albuquerque, NM (US); Kevin D. Ley, Santa Fe, NM (US); Duncan McBranch, Santa Fe, NM (US); David G. Whitten, Albuquerque, NM (US)

Correspondence Address: Supervisor, Patent Prosecution Services, PIPER RUDNICK LLP, 1200 Nineteenth Street, N.W., Washington, DC, 20036-2412, US

(21) Appl. No.: 10/712,004

(22) Filed: Nov. 14, 2003

Related U.S. Application Data
(60) Provisional application No. 60/426,034, filed on Nov. 14, 2002.

(51) Int. Cl. C12M 1/34; G01N 33/53; B05D 3/00

(52) U.S. Cl. 435/7.5; 435/287.2; 427/2.11

(57) ABSTRACT

Complexes of a biotinylated fluorescent polymer and a biotin binding protein and solid supports coated with the fluorescent polymer complexes are described. The complexes can be used as sensors for detecting biological recognition events (e.g., nucleic acid hybridization reactions or enzymatic induced polypeptide cleavage). Methods of making the complexes and methods of using the complexes for detecting the presence and/or amount of a target analyte in a sample are also described. The target analyte can be an enzyme (e.g., β-secretase) or a nucleic acid (e.g., a single stranded or double stranded nucleic acid).

This application claims priority to U.S. Patent Application Serial No. 60/426,034, filed Nov. 14, 2002, which application is incorporated herein by reference in its entirety.

This application is related to U.S. patent application Ser. No. 09/850,074, filed May 8, 2001, and U.S. patent application Ser. No. 10/621,311, filed Jul. 18, 2003. Each of these applications is incorporated by reference herein in its entirety.

The U.S. Government has a paid-up license in this invention and the right in limited circumstances to require the patent owner to license others on reasonable terms as provided for by the terms of Contract No. MDA972-00-C-006, awarded by the Defense Advanced Research Projects Agency (DARPA).

### BACKGROUND OF THE INVENTION

1. Field of the Invention

The present invention relates generally to molecular sensors and to methods for detecting molecular interactions. In particular, the present invention relates to fluorescent polymer complexes and to methods of using the complexes in biosensing applications.

2. Background of the Technology

The enzyme linked immunosorbant assay (i.e., ELISA) is the most widely used and accepted technique for identifying the presence and biological activity of a wide range of proteins, antibodies, cells, viruses, etc. An ELISA is a multi-step “sandwich assay” in which the analyte biomolecule is first bound to an antibody attached to a surface. A second antibody then binds to the biomolecule. In some cases, the second antibody is attached to a catalytic enzyme which subsequently “develops” an amplifying reaction. In other cases, this second antibody is biotinylated to bind a third protein (e.g., avidin or streptavidin). This protein is attached either to an enzyme, which creates a chemical cascade for an amplified calorimetric change, or to a fluorophore for fluorescent tagging.

Despite its wide use, there are many disadvantages to ELISA. For example, because the multi-step procedure requires both precise control over reagents and development time, it is time-consuming and prone to “false positives”. Further, careful washing is required to remove nonspecific adsorbed reagents.

Fluorescence resonance energy transfer (i.e., FRET) techniques have been applied to both polymerase chain reaction-based (PCT) gene sequencing and immunoassays. FRET uses homogeneous binding of an analyte biomolecule to activate the fluorescence of a dye that is quenched in the off-state. In a typical example of FRET technology, a fluorescent dye is linked to an antibody (F-Ab), and this diad is bound to an antigen linked to a quencher (Ag-Q). The bound complex (F-Ab:Ag-Q) is quenched (i.e., non-fluorescent) by energy transfer. In the presence of identical analyte antigens which are untethered to Q (Ag), the Ag-Q diads are displaced quantitatively as determined by the equilibrium binding probability determined by the relative concentrations, [Ag-Q]/[Ag]. This limits the FRET technique to a quantitative assay where the antigen is already well-characterized, and the chemistry to link the antigen to Q must be worked out for each new case.

Other FRET substrates and assays are disclosed in U.S. Pat. No. 6,291,201 as well as the following articles: Anne, et al., “High Throughput Fluorogenic Assay for Determination of Botulinum Type B Neurotoxin Protease Activity”, Analytical Biochemistry, 291, 253-261 (2001); Cummings, et al., A Peptide Based Fluorescence Resonance Energy Transfer Assay for Bacillus Anthracis Lethal Factor Protease”, Proc. Natl. Acad. Scie. 99, 6603-6606 (2002); and Mock, et al., “Progress in Rapid Screening of Bacillus Anthracis Lethal Activity Factor”, Proc. Natl. Acad. Sci. 99, 6527-6529 (2002).

Other assays employing intramolecularly quenched fluorescent substrates are disclosed in the following articles: Zhong, et al., Development of an Internally Quenched Fluorescent Substrate for Escherichia Coli Leader Peptidase”, Analytical Biochemistry 255, 66-73 (1998); Rosse, et al., “Rapid Identification of Substrates for Novel Proteases Using a Combinatorial Peptide Library”, J. Comb. Chem., 2, 461-466 (2000); and Thompson, et al., H“A BODIPY Fluorescent Microplate Assay for Measuring Activity of Calpains and Other Proteases”, Analytical Biochemistry, 279, 170-178 (2000). Assays have also been developed wherein changes in fluorescent polarization have been measured and used to quantify the amount of an analyte. See, for example, Levine, et al., “Measurement of Specific Protease Activity Utilizing Fluorescence Polarization”, Analytical Biochemistry 247, 83-88 (1997). See also Schade, et al., “BODIPY-α-Casein, a pH-Independent Protein Substrate for Protease Assays Using Fluorescence Polarization”, Analytical Biochemistry 243, 1-7 (1996).

There still exists a need, however, to rapidly and accurately detect and quantify biologically relevant molecules with high sensitivity.

### SUMMARY OF THE INVENTION

According to a first aspect of the invention, a method of making a sensor for detecting biological recognition events is provided. The method comprises combining a biotinylated fluorescent polymer and a biotin-binding protein in aqueous solution to form a complex, wherein the complex comprises free biotin-binding sites. A biotinylated fluorescent protein (e.g., phycoerythrin or phycobilisome) can be combined with the biotinylated fluorescent polymer and the biotin-binding protein. The complex can be disposed onto the surface of a solid support (e.g., a microsphere, a nanoparticle or a bead). The solid support can be a silica or a latex microsphere. The surface of the solid support can comprise ammonium functional groups. The biotin binding protein can be selected from the group consisting of avidin, streptavidin, and neutravidin.

The method as set forth above can further include adding to the solution a biotinylated bioconjugate comprising a polynucleotide sequence, a peptide nucleic acid sequence, or a polypeptide sequence wherein the biotinylated bioconjugate binds to free biotin binding sites in the complex. According to one embodiment, the biotinylated bioconjugate comprises a polynucleotide or peptide nucleic acid sequence and the biological recognition event is nucleic acid hybridization of the polynucleotide or peptide nucleic acid sequence of the biotinylated bioconjugate to a target analyte. The method according to this embodiment can also comprise adding a second bioconjugate comprising a quencher and a polynucleotide or peptide nucleic acid sequence to the solution, wherein the quencher is capable of amplified super-quenching of the fluorescent polymer and wherein the polynucleotide or peptide nucleic acid sequence of the second bioconjugate is capable of hybridizing to the polynucleotide or peptide nucleic acid sequence of the biotinylated bioconjugate. The polynucleotide or peptide nucleic acid sequence of the second bioconjugate can be complementary to the polynucleotide or peptide nucleic acid sequence of the biotinylated bioconjugate.

According to an alternative embodiment, the biotinylated bioconjugate comprises a polypeptide sequence and a quencher which is capable of amplified super-quenching of the fluorescent polymer and the biological recognition event is enzyme induced cleavage of the polypeptide sequence.

According to a second aspect of the invention, a sensor for detecting biological recognition events is provided which comprises a complex of a biotinylated fluorescent polymer and a biotin binding protein, wherein the complex comprises free biotin binding sites. The complex can be disposed on a surface of a solid support (e.g., a microsphere, a nanoparticle or a bead). The solid support can be a silica or a latex microsphere. A biotinylated bioconjugate comprising a polynucleotide sequence, a peptide nucleic acid sequence or a polypeptide sequence can be bound to the complex. For example, the biotinylated bioconjugate can comprise a polynucleotide or peptide nucleic acid sequence and the biological recognition event can be nucleic acid hybridization of the polynucleotide or peptide nucleic acid sequence of the biotinylated bioconjugate to a target analyte. Alternatively, the biotinylated bioconjugate can comprise a polypeptide sequence and a quencher, wherein the quencher is capable of amplified super-quenching of the fluorescent polymer and wherein the biological recognition event is enzyme induced cleavage of the polypeptide sequence. The biotin binding protein can be avidin, streptavidin, or neutravidin. The surface of the solid support can comprise ammonium functional groups. The sensor can also include a biotinylated fluorescent protein (e.g., phycoerythrin or phycobilisome) which forms a complex with the biotinylated fluorescent polymer and the biotin-binding protein.

A sensor is also provided which comprises a complex of a biotinylated fluorescent polymer, a biotin binding protein and a biotinylated bioconjugate disposed on a solid support, wherein the biotinylated bioconjugate comprises a polynucleotide or peptide nucleic acid sequence and wherein the biotinylated bioconjugate further comprises a quencher capable of amplified superquenching of the fluorescent polymer. According to this embodiment, the polynucleotide sequence is located between the quencher and the biotin on the biotinylated bioconjugate. A method of detecting the presence and/or amount of a target analyte in a sample using a sensor as set forth above is also provided which comprises combining the sample with the sensor (e.g., in solution). According to this embodiment, the target analyte comprises a polynucleotide sequence capable of hybridizing to the polynucleotide or peptide nucleic acid sequence of the biotinylated bioconjugate and hybridization of the target analyte and biotinylated bioconjugate results in increased separation of the quencher from the surface of the solid support with a concomitant increase in fluorescence.

According to a further embodiment, the sensor comprising a fluorescent polymer complex disposed on a solid support as set forth above can further comprise a biotinylated bioconjugate comprising a ligand and a biotin moiety conjugated to first and second locations on a tether wherein the ligand comprises a quencher moiety capable of amplified super-quenching of the fluorescent polymer and wherein the ligand is capable of taking part in a biological recognition event. According to this embodiment, the portion of the tether between the first and second locations has a length and a flexibility such that occurrence of the biological recognition event results in separation of the quencher from the surface of the solid support with a concomitant increase in fluorescence. The ligand can comprise a polypeptide sequence. The portion of the tether between the first and second locations can comprise a repeating unit represented by the chemical formula:

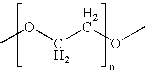

wherein n is a positive integer. A method of detecting the presence and/or amount of a target analyte in a sample using a sensor as set forth above is also provided. The target analyte can be a spore, a cell, a bacteria or a virus. A sensing system for detecting biological recognition events is also provided comprising a sensor as set forth above and a second solid support comprising a plurality of target moieties disposed on the surface thereof wherein the ligand can interact with the target moieties such that the quencher is separated from the fluorescer thereby increasing the fluorescence of the fluorescent polymer. The second solid support can be a microsphere (e.g., a silica or a latex microsphere), a nanoparticle or a bead. A method of detecting the presence and/or amount of a target analyte in a sample is also provided which comprises combining the sensing system with the sample wherein the target analyte can recognize and interact with the ligand and wherein interaction of the target analyte with the ligand results in a decrease in fluorescence. The ligand can comprise a polypeptide and the biological recognition event can be the interaction of the polypeptide of the ligand with a target analyte comprising a polypeptide.

According to a third aspect of the invention, a method of detecting the presence and/or amount of a target analyte in a sample is provided which comprises combining the sample with a biotinylated bioconjugate comprising a nucleotide sequence, a peptide nucleic acid sequence or a polypeptide sequence and a sensor comprising a fluorescent polymer complex as set forth above. When the biotinylated bioconjugate comprises a polynucleotide or peptide nucleic acid sequence, the method can further include combining the sample with a second bioconjugate comprising a quencher and a polynucleotide or peptide nucleic acid sequence wherein the quencher is capable of amplified super-quenching of the fluorescent polymer and wherein the polynucleotide or peptide nucleic acid sequence of the second bioconjugate is capable of hybridizing to the polynucleotide or peptide nucleic acid sequence of the biotinylated bioconjugate. According to this embodiment, the target analyte comprises a polynucleotide sequence which is capable of hybridizing to the polynucleotide or peptide nucleic acid sequence of either the biotinylated bioconjugate or the second bioconjugate. For example, the polynucleotide or peptide nucleic acid sequence of the second bioconjugate can be complementary to the polynucleotide or peptide nucleic acid sequence of the biotinylated bioconjugate. According to a further embodiment, the sensor and the biotinylated bioconjugate are combined such that the biotinylated bioconjugate complexes to the sensor, the sample is subsequently incubated with the sensor/biotinylated bioconjugate complex, and the second bioconjugate is subsequently added to the incubated sample. According to an alternative embodiment, the nucleotide sequence of the target analyte can comprise a double-stranded nucleic acid. According to this alternative embodiment, the method further comprises: heating the incubated sample in the presence of the second bioconjugate to a temperature sufficient to melt double-stranded nucleic acid in the sample; and cooling the sample to allow duplex formation. Duplex formation between target analyte present in the sample and the second bioconjugate results in an increase in fluorescence. Alternatively, the biotinylated bioconjugate can comprise a polypeptide sequence and a quencher and the target analyte can be an enzyme (e.g., β-secretase) capable of cleaving the polypeptide sequence.

According to a fourth aspect of the invention, a sensor for detecting a target biological species is provided which comprises: a bacterial spore or virus comprising a plurality of ligands for a receptor on a surface thereof; a fluorescent polymer or fluorescent polymer complex disposed on a surface of the bacterial spore or virus; and a plurality of bioconjugates comprising a quencher conjugated to a receptor for the ligand, wherein the receptor and ligand interact and wherein the interaction of the receptor and ligand results in amplified super-quenching of the fluorescence of the fluorescent polymer. A method of detecting the presence and/or amount of a target analyte in a sample is also provided which comprises: incubating the sample with a sensor as set forth above wherein the target analyte recognizes and interacts with the receptor and wherein interaction of the target analyte with the receptor results in an increase in fluorescence. The target analyte can be a bacterial spore or a virus comprising a plurality of ligands for the receptor on a surface thereof.

### BRIEF DESCRIPTION OF THE DRAWINGS

The present invention may be better understood with reference to the accompanying drawings in which:

FIG. 1 illustrates an assay according to the invention wherein a DNA containing QTL is used to detect a target analyte having a base sequence complementary to the DNA of the DNA containing QTL;

FIGS. 2A-2C illustrate an assay according to the invention wherein a QTL bioconjugate with a flexible tether is used to detect a multi-valent analyte;

FIG. 3 illustrates the synthesis of a multivalent antigen bead (MAB) according to the invention;

FIG. 4 illustrates the synthesis and use of a fluorescent polymer tagged inactivated target according to the invention;

FIG. 5 shows a reaction scheme for sensor fabrication according to one embodiment of the invention wherein a mixture of neutravidin and polymer repeat units is complexed and the resulting polymer-protein complex is then deposited on the surface of an ammonium functionalized microsphere through electrostactic interactions;

FIG. 6 shows an assay for DNA detection wherein quencher labeled targets compete with target for a complementary capture strand on the surface of the sensing microspheres;

FIG. 7 is a graph showing the quenching of PPE fluorescence by various oligonucleotides and mixtures of oligonucleotides; and

FIG. 8 is a graph showing mismatch analysis with a microsphere sensor loaded with a PNA-based capture strand.

### DETAILED DESCRIPTION

Bioconjugates comprising a ligand (L) for a target biological molecule tethered (T) to a quencher (Q) that associates with and quenches a fluorescent polymer (P) are disclosed in U.S. patent application Ser. No. 09/850,074, herein incorporated by reference in its entirety. These bioconjugates (designated “QTL bioconjugates”) take advantage of super-quenching of fluorescent polyelectrolytes by, for example, electron transfer or energy transfer quenching. A fluorescent polymer (P) can form an association complex with a QTL bioconjugate, usually one with a charge opposite that of the fluorescent polymer. The QTL bioconjugate includes a quencher (Q) linked through a covalent tether to a ligand (L) that is specific for a particular biomolecule. The association of the ligand of the QTL bioconjugate with the biomolecule either separates the QTL bioconjugate from the fluorescent polymer, or modifies its quenching in a readily detectable way, thus allowing sensing of the biomolecule by a change in fluorescence. In this manner, the biomolecule can be detected at very low concentrations.

It has also been demonstrated that coating the fluorescent polymer on a support such as a latex or silica bead or nanoparticle can lead to an increase in super-quenching and a concomitant decrease in fluorescence changes due to nonspecific interactions with macromolecules such as proteins or nucleic acids. As a result, assays have been devised that employ fluorescent polymer and receptor co-located on the same particle such that the QTL interaction with the fluorescent polymer is mediated by association of the L portion of the QTL conjugate with a specific receptor. Assays of this type are disclosed in U.S. patent application Ser. No. 10/098,387, filed Mar. 18, 2002, which is herein incorporated by reference in its entirety. These assays are typically competition assays wherein the analyte either consists of, or contains a sequence L, recognized by the surface associated receptor. Binding of L with the receptor therefore produces little or no change in fluorescence from the polymer or polymer ensemble. Binding of the QTL by association with the receptor, however, leads to a quench of the fluorescence.

1. Pre-Formed Polymer-Protein Complexes for Sensing in Solution and in Supported Formats.

As described above, QTL-polymer superquenching assays have been constructed by co-locating fluorescent polymers, such as a polyanionic polyphenylene ethynylene (1):

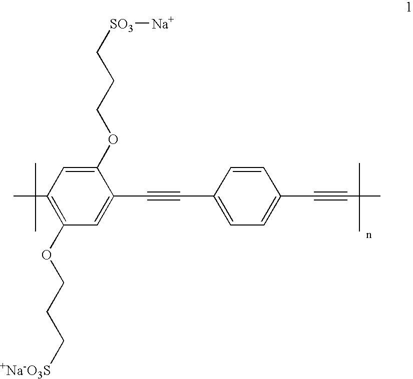

or a biotinylated polyphenylene ethynylene (2):

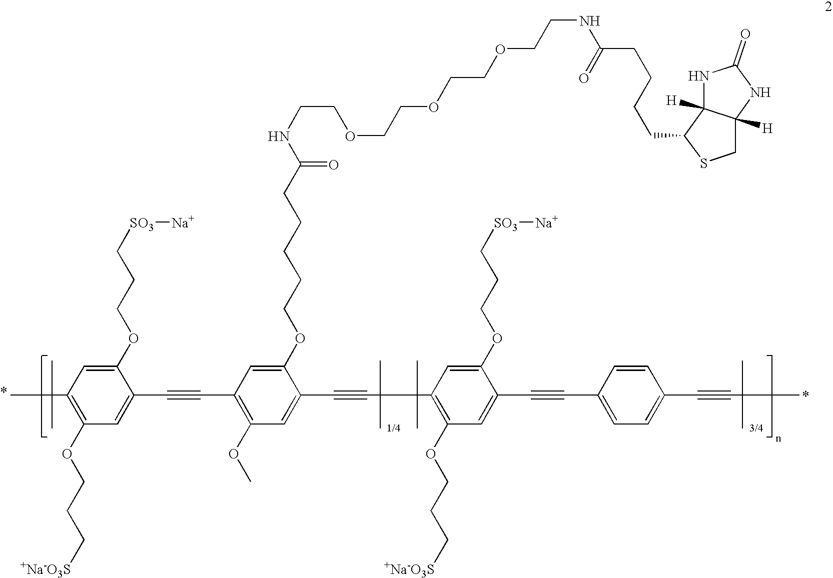

and a receptor on a support such as a latex or silica bead or nanoparticle. Typically the receptor may be an antibody; protein, oligonucleotide or other ligand. The receptor and/or the polymer can be affixed to the support through biotin-avidin association. One of the biotin-binding proteins (avidin, neutravidin or streptavidin) can be covalently linked to the support prior to addition of polymer or receptor.

According to the present invention, an alternative means of co-locating fluorescent polymer and acceptor is provided that involves the initial complexation of a biotinylated fluorescent polymer (e.g., polymer 2) with a biotin-binding protein in solution. Polymer 2 contains several available biotins yet can only bind to one or at most two of the four biotin-binding sites each of these proteins have available. This is in part due to the “rigid rod” nature of large segments of the PPE polymer.

However, addition of a biotinylated fluorescent polymer, such as polymer 2 above, to a biotin binding protein such as neutravidin in aqueous solution results in cross-linking of the polymer by the protein. For polymer 2, this cross-linking is accompanied by a moderate increase in the polymer fluorescence and a significant increase in ensemble size as indicated by light scattering. Depending upon the ratio of biotin-binding protein to polymer, as well as the precise sequence of addition, the resulting ensemble of the biotinylated fluorescent polymer and the biotin-binding protein can contain a moderate number of free biotin-binding sites that can be used to affix specific biotinylated receptors such as antibodies, proteins, oligonucleotides or peptides. When the biotin functionalized receptor contains a quencher (either as part of the receptor or as a receptor-QTL complex) efficient quenching of the polymer fluorescence can occur.

The biotin binding protein/biotinylated fluorescent polymer ensemble can be coated onto a solid support. For example, a neutravidin:polymer 2 ensemble (i.e., 1 neutravidin: 15 polymer repeat units) was coated onto latex microspheres functionalized with quaternary ammonium groups. The resulting microspheres were highly fluorescent. This fluorescence could be specifically quenched by the addition of a biotin-quencher conjugate. In contrast, addition of the quencher not containing a biotin resulted in minimal non-specific quenching. The quenching was slightly enhanced over that observed for the same composition solution-phase neutravidin:polymer 2 ensemble.

A preformed polymer-biotin-binding protein complex thus affords the basis for sensing applications either in solution or in supported formats. In both “platforms” the complexes offer certain advantages. First, the close proximity of receptor and polymer is assured. Second, the ensemble is less subject to nonspecific interactions with reagents such as proteins, small organic molecules and inorganic ions. Additionally, a wide tuning of the assay is also possible. For example, one or more of the following parameters can be varied: the ratio of biotin-binding protein to biotinylated polymer; the biotin density on the polymer; the sequence of addition; or the specific biotin-binding protein used. In this manner, the assay can be tailored for a specific application. Additionally, the overall charge of the complex may be tuned by varying the charged side groups on the polymer or by varying the biotin binding protein. The complex may thus be chosen to enhance or eliminate non-specific binding to other proteins, non-specific binding to other biomolecules (e.g., DNAs or PNAs), or non-specific binding to charged or neutral surfaces.

2. Assays for single stranded and duplex DNA based on Fluorescent Polymer Superquenching.

Sensing with fluorescent polyelectrolytes may be applied to oligonucleotide-oligonucleotide recognition. For example, a QTL-based sensing of single stranded DNA has recently been reported [Kushon, et al., Langmuir, 18, 7245-7249 (2002)]. In the simplest case, a single strand “target” DNA sequence may associate with a complementary “capture” single strand such that the fluorescence of polymer or polymer ensemble is modulated (quenched or enhanced). One approach involves the use of a biotinylated capture strand of DNA, complementary to a “target” sequence. Competition assays have been developed using a quencher-tagged target (DNA-QTL) in known amount in the presence of an unknown amount of the target analyte. In these assays the biotinylated capture strand was associated with a bead support containing a fluorescent polyelectrolyte and a biotin binding protein such as avidin, streptavidin or neutravidin. Association of the biotinylated capture strand with the beads (via biotin-avidin association) resulted in little or no change in the polymer fluorescence. Likewise, association of the biotinylated capture strand-target analyte duplex with the beads resulted in little or no fluorescence change. However association of the biotinylated capture strand-DNA-QTL duplex with the beads or the DNA-QTL with previously bound biotinylated capture strand resulted in a strong quenching of the polymer fluorescence.

A direct competition between the DNA-QTL and the target analyte for biotinylated capture strand pre-associated with the beads led to low assay sensitivity due to the faster (kinetic) association of the DNA-QTL with the capture strand (compared with the unlabeled analyte). However a stepwise association of the analyte with the bead-bound capture strand, followed by addition of the DNA-QTL afforded a sensitive and yet simple quantitative assay. A similar sensitive assay was also obtained by pre-incubating the biotinylated capture strand, DNA-QTL and analyte single strand DNA and then exposing this mixture to the fluorescent polymer coated beads. For both of the latter assays the level of fluorescence increases with increase in concentration of the single strand analyte DNA.

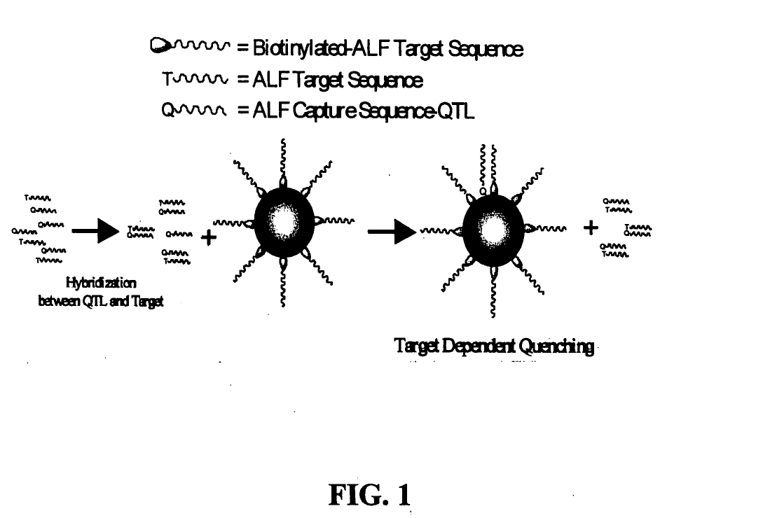

The above described assays employ single stranded DNA and involve the use of a DNA-QTL that contains the same base sequence as the target analyte. An alternative assay format is shown in FIG. 1. This assay format involves using a DNA-QTL that has a base sequence complementary to the target analyte.

As shown in FIG. 1, an energy transfer or electron transfer quencher can be covalently linked to one end of the strand to generate the DNA-QTL. As also shown in FIG. 1, a biotinylated strand having the same sequence as the target analyte and a biotin on one end of the strand can be employed as the capture strand. Association of the biotinylated capture strand with the fluorescent polymer-coated beads results in little or no change in the level of fluorescence from the polymer. Duplex formation between the DNA-QTL and the bead-bound capture strand, however, results in a quenching of the polymer fluorescence due to the close association between the polymer and the quencher on the DNA-QTL.

To accomplish an assay for single strand analyte DNA, the analyte (unknown level) and DNA-QTL can be mixed with a suspension of the beads containing the biotinylated target. Duplex formation between the target analyte and DNA-QTL removes “free” DNA-QTL, thereby inhibiting the quenching of the polymer that would occur in the absence of the target. In this manner, a simple and homogeneous quantitative assay for the single strand analyte can be provided.

The above described assay materials can also provide a simple and homogeneous format for sensing a target analyte present as a duplex. For example, a sample containing an analyte and duplex DNA-QTL having a base sequence complementary to the analyte can be added to a solid support (e.g., a suspension of beads) containing co-located fluorescent polymer and biotinylated capture reagent followed by heating to a temperature sufficient to provide for “melting” of the duplex. This leads, after returning the mixture to ambient temperature, to pairing of the DNA-QTL with the single strand analyte and to an attenuation of fluorescence quenching proportional to the level of the target strand in the sample.

Assays similar to those previously reported and described above can be constructed using a biotinylated capture strand of peptide nucleic acids (i.e., biotinylated PNA). The biotinylated PNA exhibits similar selectivity in pairing with complementary sequences of target analyte DNA or DNA-QTL's but affords a stronger duplex and thus can provide even greater sensitivity in assays for single strand target analyte. An advantage with the biotinylated PNA as the capture strand is that the greater strength of the DNA-PNA association provides the basis for an ambient temperature homogeneous assay for duplexed target by strand-invasion.

Another alternative method of DNA detection involves the use of a biotinylated DNA-QTL. The biotin and the quencher in the conjugate are placed at opposing ends. When exposed to a polymer-coated microsphere that bears biotin-binding proteins, the biotin-DNA-QTL becomes attached to the surface through the biotin. Additionally, due to the general hydrophobicity of the quenchers that are employed, the quencher labeled terminus folds back onto the surface, allowing the quencher to quench the polymer that lies on the surface. However, in the presence of target strand, the biotin-DNA-QTL is hybridized into a DNA duplex. DNA duplexes are known to be relatively rigid compared to single stranded DNA. Therefore, formation of the duplex can result in an increased distance between the quencher and the surface since the biotin-DNA-QTL cannot fold back onto the surface as readily with the DNA hybridized to the target. As a result, the level of quenching can be reduced.

3. Sensing Format Based on the Use of Long, Flexible Tethers (e.g., Hydrophilic Polymeric Tethers)

As set forth above, the QTL conjugate used in biosensing based on the quenching/unquenching of fluorescent polymers or polymer ensembles typically consists of three components: the quencher (O); the tether (T); and the ligand or receptor (L). The degree of superquenching, whether by energy transfer or electron transfer, is dependent on proximity of the quencher to the fluorescent polymer or polymer ensemble. The degree of sensitivity of the biological recognition event that is sensed is typically dependent on a coupling of the recognition event with a change in the distance separating the quencher and polymer ensemble.

In the initial approaches to biosensing based on polymer/QTL super-quenching interactions, the polymer (in solution, or bound to supports such as microspheres or nanoparticles) associates with the QTL by virtue of nonspecific interactions (generally a combination of Coulombic attraction and hydrophobic interactions). In a fluorescence “turn-off” assay, association of the QTL, released in the biological recognition event, with the polymer results in a quenching of the fluorescence. Alternatively, association of the QTL with a specific receptor can result in separation of pre-associated polymer and QTL and lead to a fluorescence “turn-on” sensing. This assay platform can be used in both direct and competition assays, depending on the target analyte and synthetic QTL.

In an alternative sensing platform, both the fluorescent polymer and receptor (i.e., the receptor for the ligand “L” of the QTL bioconjugate) are co-located on a solid support such as a micron-sized or sub micron-sized latex bead, a silica microsphere, nanoparticle or surface. In this case specific association of the QTL with the receptor leads to quenching of fluorescence while release of the QTL leads to a turn on of fluorescence. In both of the sensing approaches discussed above, the QTL conjugate generally employs a tether of minimum length so as to provide for close proximity of fluorescent polymer and both the quencher and ligand portions of the QTL, when the QTL is associated with the polymer or polymer ensemble.

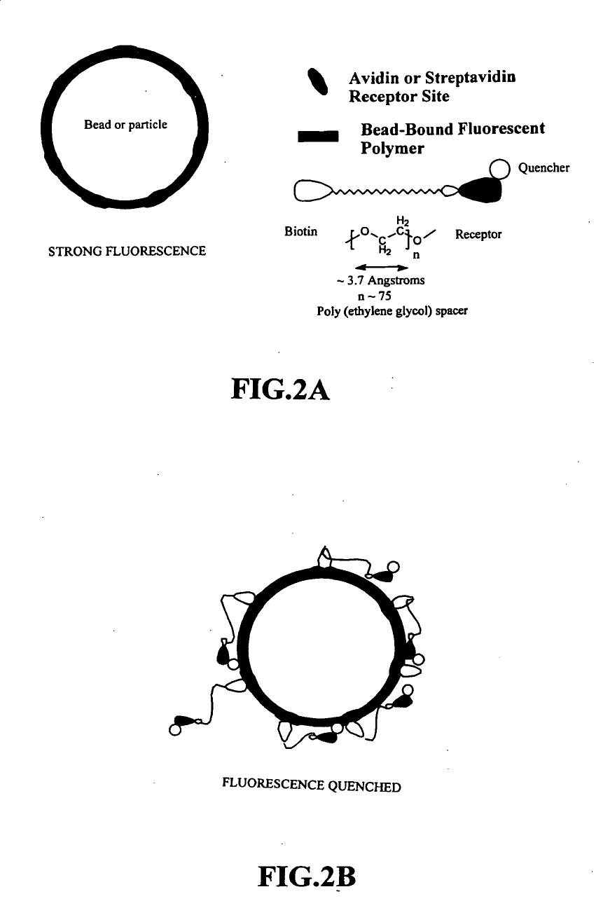

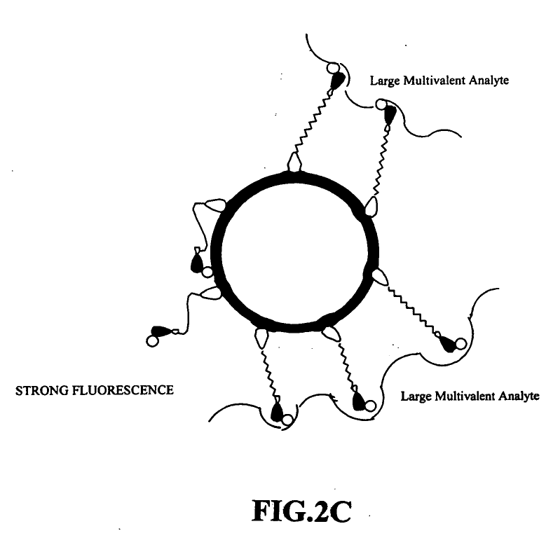

An alternate approach incorporates a QTL conjugate with a long flexible tether. As shown in FIGS. 2A-2C, construction of a “flexible” tether separating a biotin “connector” from a recognition molecule bearing a quencher leads to a QTL that can be associated with a bead “platform” containing a biotin-binding protein and a fluorescent polymer.

As shown in FIGS. 2A-2C, a solid support (a bead is shown) coated with a fluorescent polymer and having available avidin or streptavidin receptor sites can be complexed with a biotinylated quencher having a long flexible tether. As a result, fluorescene is quenched (FIG. 2B). The presence of an analyte which binds the recognition molecule, however, can remove the quencher from the fluorescent support resulting in an increase in fluorescence (FIG. 2C).

The flexible tether can exist in a variety of conformations. In a preferred embodiment, the flexible tether consists of a poly (ethylene glycol) (i.e., PEG) linear chain as shown in FIG. 2A. In one example a biotin is separated from a receptor by a PEG tether that has ˜75 repeat units. If this chain were in a fully extended conformation, the distance between the biotin connector and the receptor would be ˜278 Angstroms.

In aqueous media the PEG chain should be somewhat collapsed and, in the collapsed or coiled state, the quencher-labeled receptor may be brought into relatively close proximity of the bead-bound fluorescent polymer. This can result in quenching of fluorescence from polymer regions that may be relatively far removed (on the surface of the bead) from the biotin-binding protein site to which the biotin of the QTL is associated. The degree of interaction between the quencher-receptor at the end of the chain and the fluorescent polymer on the surface may be adjusted by varying the charge on the surface and the quencher-receptor, by varying the hydrophobicity of the quencher-receptor or by reagents added to the suspension.

The flexible chain is preferably long enough that when it is fully extended away from the surface, the quencher-labeled receptor is too far from the polymer to permit significant quenching. Since the association between the receptor-quencher and the fluorescent polymer on the surface of the bead is weak, addition of an analyte that is large can result in removal of the receptor-quencher and extension of the PEG to a distance outside of the quenching radius of the polymer. For a large, multivalent analyte the sensing can be amplified by removal of multiple receptor-quenchers from the same or multiple beads. Thus this assay format is particularly suitable to relatively large analytes such as spores, cells, bacteria or viruses.

4. Multivalent Antigen Beads as Basis for QTL Biosensing.

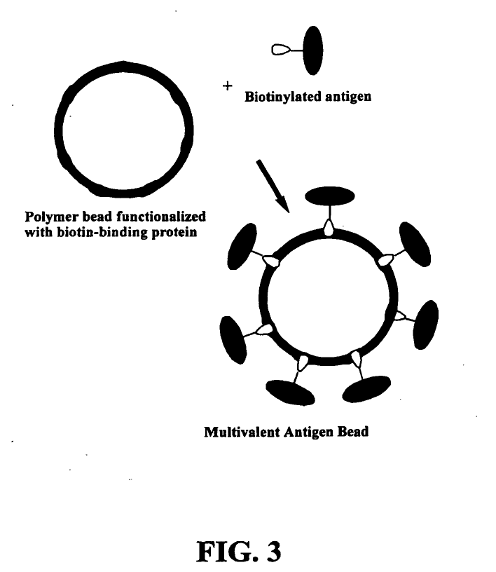

According to a further embodiment of the invention, assays may be constructed using the same beads and conjugates with long flexible tethers described above further comprising two components that interact differently in the presence of a target protein analyte. In this case, the assays are particularly suitable for small protein analytes that do not elicit the response indicated in Section 3 above, but which can bind to the receptor-quencher ensemble without leading to its removal from the fluorescent polymer. In this case one of the components is the fluorescent polymer coated bead containing a biotin-binding protein and the biotin-flexible tether-receptor-quencher “QTL component” described in Section 3 above. The second component can be a polymer bead or microsphere whose surface is “decorated” with multiple copies of the target antigen recognizing the receptor (i.e., a “multivalent antigen bead” or MVAB). An MVAB is shown in FIG. 3.

As shown in FIG. 3, a biotinylated antigen can be complexed with a polymer bead functionalized with biotin binding protein to form a multivalent antigen bead according to the invention.

Addition of the multivalent antigen beads to suspensions of the beads containing the biotin-flexible tether-receptor-quencher leads to a turn on of fluorescence from the polymer by removal of the quencher-receptor from the surface of the bead. Subsequent addition of target analyte results in a quenching of the fluorescence by competition for the receptor and displacement of the MVAB. This assay can be conducted in a direct competition mode where a known amount of the MVAB and an unknown amount of target protein analyte are added simultaneously to a suspension of the beads containing the fluorescent polymer. The level of fluorescence quenching provides a direct measure of the concentration of analyte. The assay can also be conducted as a displacement competition assay by sequential treatment of the fluorescent polymer-receptor coated beads with either target analyte followed by MVAB or vice versa.

5. QTL Sensing by Fluorescent Polymer or Polymer-Ensemble-Tagged Targets.

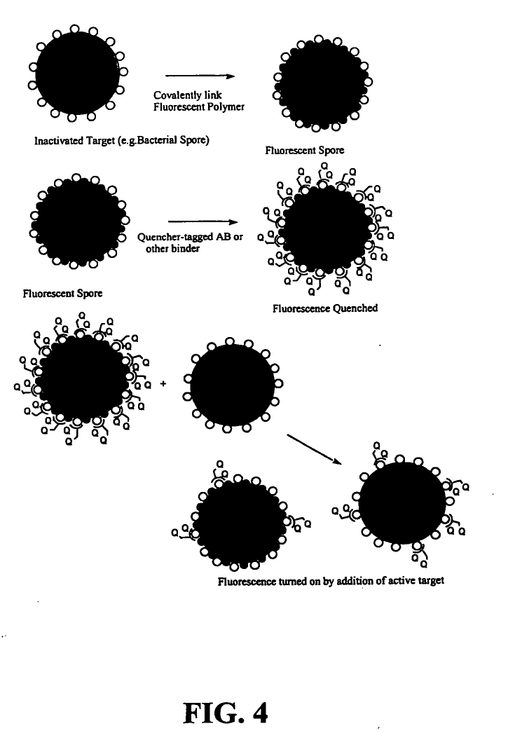

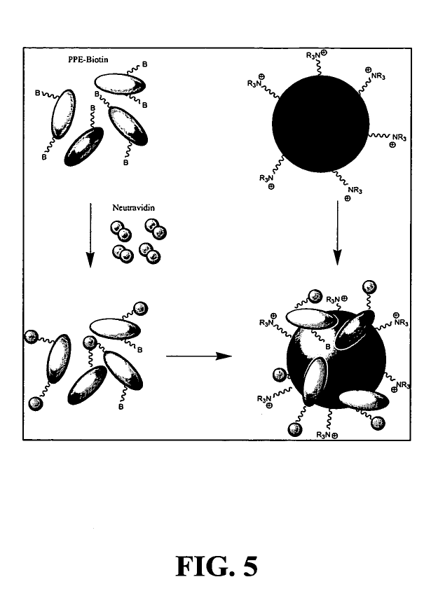

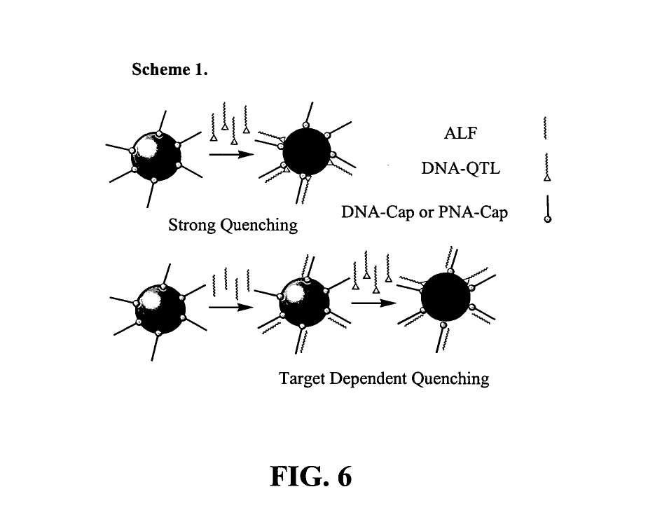

Large and robust biological species such as bacterial spores and viruses that have a repeated pattern on their surface consisting of both antigenic and chemically reactive sites afford an alternative polymer super-quenching assay. As shown schematically in FIG. 4, an inactivated target of this type can be reacted to covalently link or otherwise attached to the surface a fluorescent polymer or fluorescent polymer ensemble.

As shown in FIG. 4, a fluorescent polymer can be covalently linked to an inactivated target (e.g., a bacterial spore) to form a functionalized inactivated target. A fluorescent spore is shown in FIG. 4. The level of attachment can be controlled such that sites for binding of receptors to the target remain accessible.

As shown in FIG. 4, the functionalized inactivated target (i.e., the fluorescent spore) is highly fluorescent. Addition of receptor-quencher QTL bioconjugates (e.g., where the receptor may be an antibody, an antibody fragment or other binding reagent such as a peptide or other small molecules binder) results in binding to the fluorescent target with a quenching of its fluorescence. As shown in FIG. 4, each tagged target can accommodate several molecules of receptor-quencher conjugate. As also shown in FIG. 4, the addition of unlabeled target results in a “dilution” of receptor binding sites and a removal of the receptor-quencher conjugates from the fluorescent tagged targets. As a result, an increase in fluorescence can be observed.

The sensitivity of the above described assay may be tuned by adjusting the level of coating of the fluorescent polymer on the target, tuning the structure of the conjugate and its affinity for tagged and un-tagged target. As indicated with several previous QTL polymer superquenching assays, the actual competition may be carried out in several different modes, ranging from pre-incubation of labeled target with the quencher-binder QTL to direct mixing of the QTL, target and labeled target.

Pre-Formed Polymer-Protein Complexes for Sensing in Solution and Supported Formats

### EXAMPLE 1

### Preparation of a Polymer-Protein Complex for Sensing in Solution

A QTL solution sensor (“Sensor SS”) was prepared by mixing together 56.5 nmol of Avidin (Biotin binding protein, BBP) and 848 nmol of biotinylated PPE polymer (1) in a total volume of 11.3 mL and incubating at CRT for 24 hours. The polymer and the BBP combine with each other through the biotin-avidin interaction to form stable entities. The solution sensor thus prepared was diluted appropriately with buffer at the beginning of each experiment. The structure of polymer (1) is shown below:

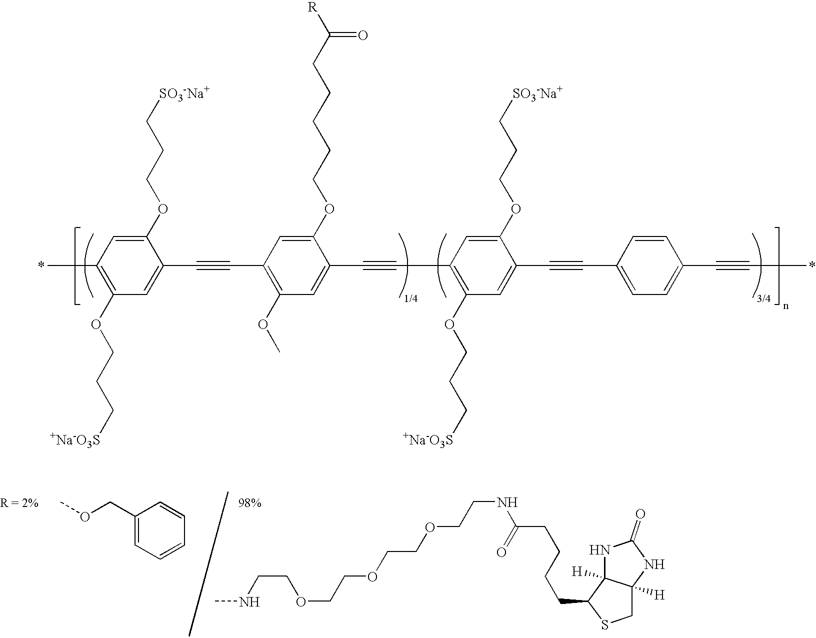

### Structure of Polymer 1

### EXAMPLE 2

### Adaptation of Pre-Formed Protein-Polymer Complexes for Sensing at a Solid-Solution Interface

In this example, polymer-protein ensembles were coated onto quaternary ammonium functionalized polystyrene microspheres (MS), 0.55 micron diameter (from Interfacial Dynamics Corporation), by a two step procedure. In step one, a predetermined amount of polymer (1) in solution is added to a solution of Neutravidin (another BBP) so that the final ratio of polymer repeat units (PRUs) to BBP is 5:1. This solution is incubated under ambient conditions for 30 minutes. In the second step, the polymer/protein mixture is added to the polystyrene microspheres and incubated for 2 hours at pH=7, then diafiltered and exchanged into phosphate buffered saline. Difference fluorescence spectroscopy was employed to quantify the polymer and protein coating densities.

The estimated polymer coating density is 4.75×10⁶ PRU/MS, and the estimated protein coating density is. 9.5×10⁵ Neutravidins/MS for PPE-B. Upon coating of the polymer/protein mixture onto the surface of the microspheres, the spheres were determined to have ˜1.3×10⁵ biotin binding sites per sphere, as determined from binding experiments employing a fluorescein labeled biotin derivative.

FIG. 5 shows a reaction scheme for sensor fabrication as set forth above wherein a mixture of neutravidin and fluorescent polymer is complexed and the resulting complex coated onto a solid support. As set forth above, the ratio of polymer repeat units to neutravidin can be 5:1. As shown in FIG. 5, the complex can be deposited onto the surface of an ammonium functionalized microsphere through electrostactic interactions.

### EXAMPLE 3

### Sensing for Enzyme Activity Using a QTL Solution Sensor (“Sensor SS”) as Prepared in Example 1

To 5 μL of a 400 nM solution of BSEC-1 (structure shown below) in assay buffer in a 384-well plate was added 30 ng of β-secretase enzyme dissolved in 5 μL of assay buffer. BSEC-1 has a peptide structures as set forth below:

(QSY7)-T-E-E-O-S-E-V-N-L-D-A-E-F-(K-Biotin)-OH SEQ ID NO: 1

wherein “QSY7” and “Biotin” are represented by the following formulae:

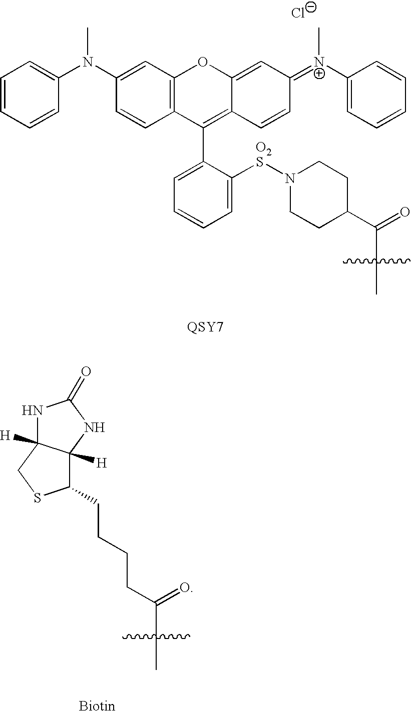

The mixture was made in triplicate and incubated for 30 minutes at CRT. The control wells contained only peptide and no enzyme. After incubation, a 100-fold dilution of the above solution sensor was added at 20 μL to each well. The plate was shaken inside the microplate reader and the wells were probed by exciting the polymer at 440 nm and measuring the emission intensity at 530 nm using a 475 nm cut-off filter. The control wells gave an average RFU value of 5,400±200 and the sample wells containing enzyme gave an average RFU value of 8,350±200. The difference in fluorescence was a measure of enzyme activity.

Although BSEC-1 is disclosed above, other polypeptides can also be used in assays for β-secretase enzyme activity. For example, according to an alternative embodiment of the invention, BSEC-3 can be used in an assay for β-secretase enzyme activity. BSEC-3 has a polypeptide structure as set forth below:

(AZO)-T-E-E-I-S-E-V-N-L-D-A-E-F-(K-Biotin)-OH SEQ ID NO: 2

In the above peptide structures, “QSY7” and “Biotin” are defined as set forth above and “AZO” has a structure represented by the following formula:

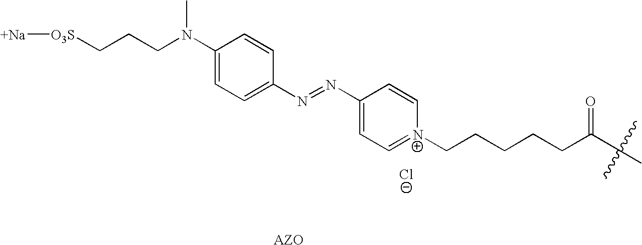

### EXAMPLE 4

### Assay Using Biotin-R-Phycoerythrin Which can Complex with the Polymer-Protein Complex Using Additional Biotin-Binding Sites on the BBP

The assay performance from Example 3 was improved by doping a QTL solution sensor as set forth in Example 1, above, with a small amount of Biotin-R-Phycoerythrin (BRPE). The resulting solution sensor (“Sensor YY”) was made at the beginning of each experiment by incubating a 200-fold dilution of the master stock of “Sensor SS” with BRPE in a ratio that would provide 250 fmol of the latter in 40 μL of the mixture. To 5 μL of a 300 nM solution of BSEC-3 in assay buffer was added 30 ng of β-secretase enzyme in 5 μL of assay buffer. BSEC-3 has a polypeptide structure as set forth above. After incubating the control and sample mixtures for 30 minutes at CRT, 40 μL of the doped solution sensor (Sensor YY) was added to each well. The plate was shaken inside the plate reader for 60 seconds and the wells were probed for fluorescence intensity by exciting the polymer at 440 nm and measuring the emission intensity at 576 nm using a 475 nm cut-off filter. The control wells gave an average RFU value of 5,200±200 and the sample wells containing enzyme gave RFU of 14,500±200. This observed difference was a measure of enzyme activity.

### EXAMPLE 5

### Assays for Single Stranded and Double Stranded DNA Based on Fluorescent Polymer Superquenching

The following data demonstrates the specificity of the QTL assay for DNA detection. The approached that was used involves the competition of quencher labeled target with target for a complementary capture strand on the surface of the sensing microspheres.

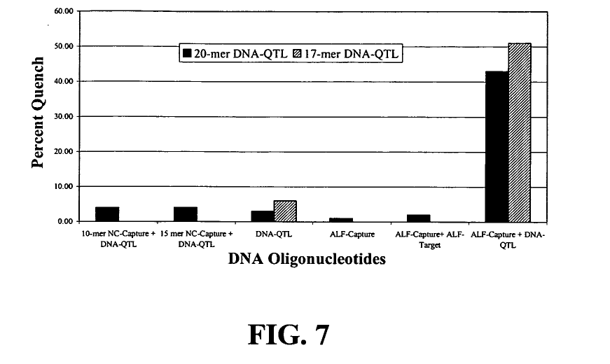

FIG. 7 is a graph showing the quenching of PPE fluorescence by various oligonucleotides and mixtures of oligonucleotides. As can be seen from FIG. 7, minimal quenching is observed due to non-specific interactions of the DNA-QTLs and the microsphere surfaces. In contrast, the specific interaction of the DNA-QTL conjugates and a capture strand resulted in significant quenching above that of the non-specific quenching. The capture strand used (i.e., ALF-Capture, structure shown below) was a biotinylated DNA capture strand bearing a sequence complementary to a region of the sequence coding for Anthrax Lethal Factor (ALF).

The results are shown in FIG. 7 using a 17-mer and a 20-mer DNA-QTL. All experiments shown in FIG. 7 were performed at 25° C. in a 96-well plate (200 mL Vₜper well). In each case, 20 pmoles of the oligonucleotide or mixtures of oligonucleotides were added.

The polypeptides referenced in FIG. 7, are defined as follows:

| | |
|---|---|
| ALF-Capture: | |
| 5′-Biotin-TAA ATA CCA TTA AAA ATG | SEQ ID NO: 3 |
| CA-3′ | |
| ALF-Target: | |
| 5′-TGC ATT TTT AAT GGT ATT TA-3′ | SEQ ID NO: 4 |
| DNA-QTL (20-mer): | |
| 5′-TGC ATT TTT AAT GGT ATT TA- | SEQ ID NO: 5 |
| QSY7-3′ | |
| DNA-QTL (17-mer): | |
| 5′-ATT TTT AAT GGT ATT TA-QSY7-3′ | SEQ ID NO: 6 |

wherein “Biotin” and “QSY7” are defined as set forth above. Non-complementary DNA oligonucleotides are denoted “NC” in FIG. 7.

As can be seen from FIG. 7, the presence of both the ALF-Capture strand and the DNA-QTL resulted in a significant increase in quenching. This increase in quenching is a result of hybridization of the DNA-QTL and the ALF-Capture strand. Moreover, the ALF-capture strand, which is biotinylated, forms a complex with the fluorescent polymer and biotin binding protein on the surface of the microsphere. Hybridization of the DNA-QTL and the ALF-Capture strand therefore brings the quencher into close proximity with the fluorescent polymer resulting in amplified superquenching.

### EXAMPLE 6

### Use of Supported Polymer-Protein Complex for Detection of Single Nucleotide DNA Mismatches

The following example demonstrates that a sensor (e.g., a sensor as described in Example 2) can be used to detect the presence of even single nucleotide DNA mismatches. The approach used in this example involves the competition of quencher labeled target with target for a complementary capture strand on the surface of sensing microspheres.

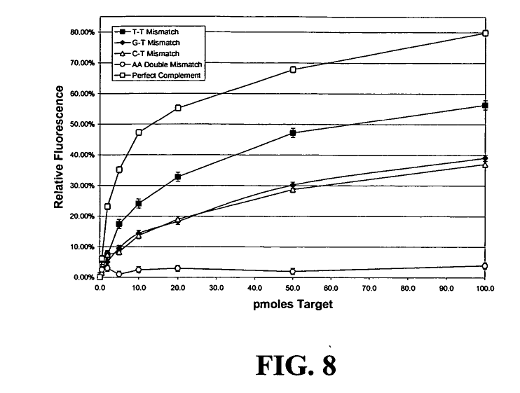

FIG. 8 is a graph showing mismatch analysis with a microsphere sensor loaded with a PNA-based capture strand (denoted “PNA-Cap”) having a structure shown below. The experiments were performed at 40° C. with a total well volume of 200 μL.

The polypeptides used in the above experiments and referenced in FIG. 8, are defined as follows:

| | |
|---|---|
| ALF Target: | |
| 5′-TGC ATT TTT AAT GGT ATT TA-3′ | SEQ ID NO: 7 |
| G—T Mismatch: | |
| 5′-TGC ATT TTT GAT GGT ATT TA-3′ | SEQ ID NO: 8 |
| T—T Mismatch: | |
| 5′-TGC ATT TTT TAT GGT ATT TA-3′ | SEQ ID NO: 9 |
| C—T Mismatch: | |
| 5′-TGC ATT TTT CAT GGT ATT TA-3′ | SEQ ID NO: 10 |
| Double Mismatch: | |
| 5′-TGC ATA TTT AAT GGA ATT TA-3′ | SEQ ID NO: 11 |
| DNA-QTL: | |
| 5′-ATT TTT AAT GGT ATT TA-QSY7-3′ | SEQ ID NO: 12 |
| PNA-Capture: | |
| Biotin-TAA ATA CCA TTA AAA-Lys-NH₂ | SEQ ID NO: 13 |

In the above formulae, “Biotin” and “QSY7” are defined as set forth above.

As can be seen from FIG. 8, increases in the relative fluorescence with increasing amounts of target were observed for all targets except the AA double mismatch target. However, the amount of increased fluorescence observed with increasing target amount was much higher for the perfect complement target.

The solid support can be made from any material suitable for use in a bioassay. The solid support can also be of any size, shape and form. The material from which the solid support is made and the size, shape and form of the solid support can be varied based on the requirements of the assay being conducted. Exemplary solid supports include, but are not limited to, microspheres, nanoparticles and beads. For example, silica or latex microspheres can be used as a solid support.

The surface of the solid support can comprise functional groups. The solid support can be made from a material comprising functional groups or, alternatively, the surface of a solid support which does not contain such groups can be functionalized to contain such groups using art recognized techniques. As set forth above, the surface of the solid support can comprise ammonium functional groups (e.g., the surface of the solid support can be functionalized to comprise ammonium functional groups). The solid support surface can also comprise or be functionalized to comprise other functional groups including, but not limited to, charged reactive groups, neutral reactive groups, and carboxylate reactive groups.

The fluorescent polymer used in the complex can be a conjugated polymer that is either neutral, positively or negatively charged, or zwitter-ionic. The fluorescent polymer can also be a side-chain polymer comprising a non-conjugated backbone with pendant fluorescent dyes that exhibit J-type aggregation behavior. Structures of exemplary fluorescent polymers are given below:

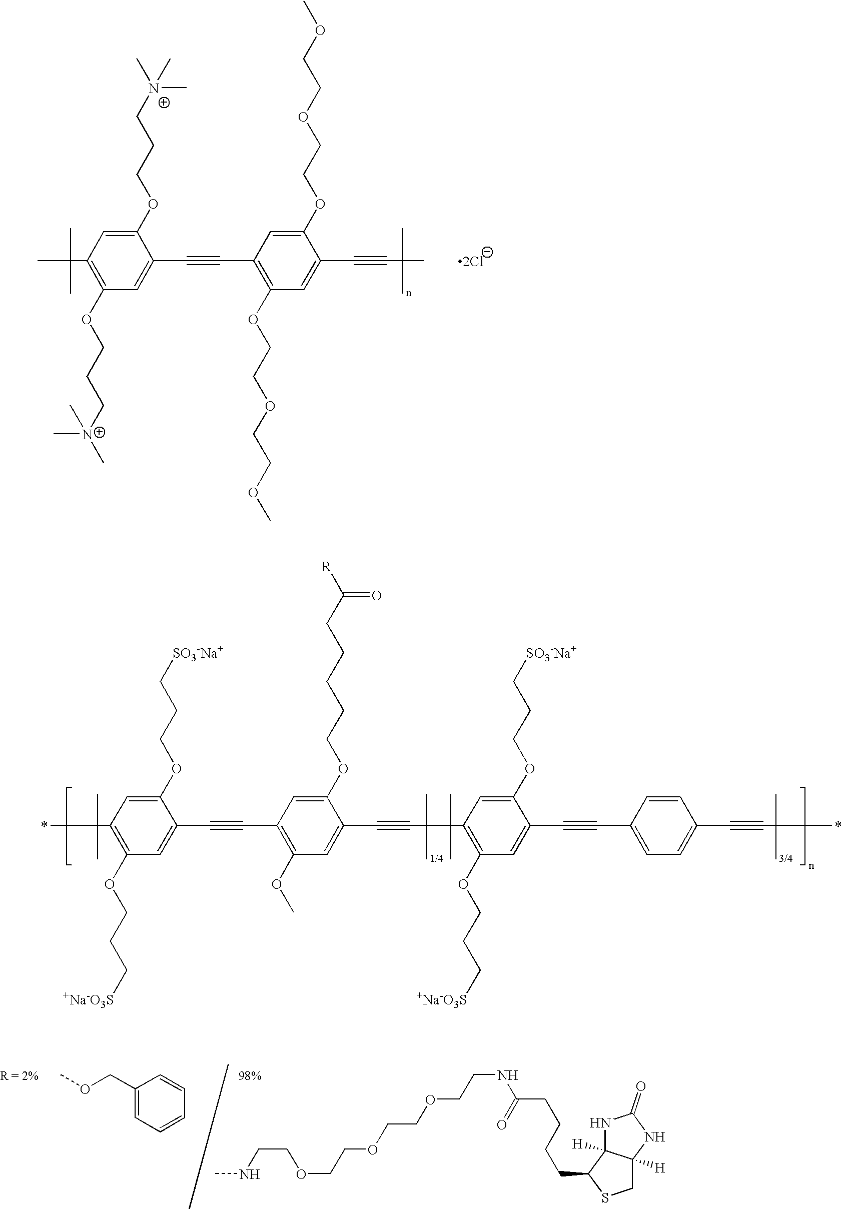

Any moiety which can absorb the radiative energy from the excited fluorescent polymer to quench the fluorescence can be used as a quencher. Exemplary quenchers include, but are not limited to, the following species: neutral, positively or negatively charged or zwitter-ionic, non-fluorescent or fluorescent, organic, inorganic, organometallic, biological or polymeric, or energy or electron-transfer species. According to one embodiment of the invention, the quencher is a non-fluorescent small molecule dye such as a QSY-7 or an Azo dye as set forth above. According to one embodiment, the quencher is capable of amplified quenching (i.e., superquenching) of the fluorescent polymer. According to a further embodiment, the quencher is capable of re-emitting as fluorescence the absorbed radiative energy from the fluorescer.

The fluorescent polymer complex can further comprise a biotinylated fluorescent protein. The biotinylated fluorescent protein can bind to free biotin binding sites of the biotin binding protein. Exemplary fluorescent proteins include, but are not limited to, phycoerythrin and phycobilisome. For example, the biotinylated fluorescent protein can be Biotinylated R-Phycoerythrin (BRPE). In the presence of the fluorescent protein, the excited chromophores of the fluorescent polymer can transfer their energy to the fluorescent protein molecules in the complex. The fluorescent protein molecules can then re-emit that energy more efficiently. For example, the use of a fluorescent polymer complex comprising BPRE can result in a sharp, red-shifted fluorescent signal. The fluorescent emissions from the complex can then be quenched when a bioconjugate comprising a quencher becomes associated with the complex (e.g., when a biotinylated bioconjugate comprising a quencher binds to the complex or when a second bioconjugate comprising a polynucleotide or peptide nucleic acid sequence and a quencher hybridizes to a capture strand associated with the complex).

While the foregoing specification teaches the principles of the present invention, with examples provided for the purpose of illustration, it will be appreciated by one skilled in the art from reading this disclosure that various changes in form and detail can be made without departing from the true scope of the invention.

What is claimed is:

1. A method of making a sensor for detecting biological recognition events comprising:
combining a biotinylated fluorescent polymer and a biotin-binding protein in aqueous solution to form a complex, wherein the complex comprises free biotin-binding sites.

2. The method of claim 1, further comprising:
combining a biotinylated fluorescent protein with the biotinylated fluorescent polymer and the biotin-binding protein.

3. The method of claim 2, wherein the fluorescent protein is phycoerythrin or phycobilisome.

4. The method of claim 1, further comprising disposing the complex onto the surface of a solid support.

5. The method of claim 1, wherein the fluorescent polymer comprises a repeating unit represented by the following general formula: 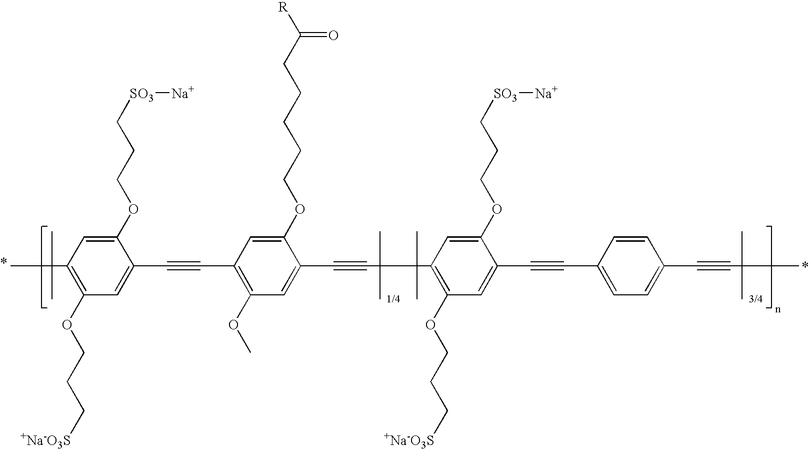
wherein n is a positive integer; and
wherein the substituent “R” is represented by the formula: 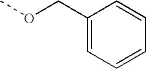
and/or the formula:

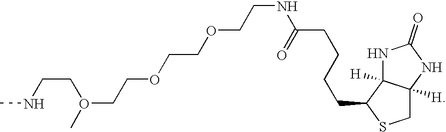

6. The method of claim 4, wherein the solid support comprises a microsphere, a nanoparticle or a bead.

7. The method of claim 4, wherein the surface of the solid support comprises a functional group selected from the group consisting of ammonium functional groups, carboxylate functional groups, charged reactive groups, and neutral reactive groups.

8. The method of claim 1, wherein the biotin binding protein is selected from the group consisting of avidin, streptavidin, and neutravidin.

9. The method of claim 1, the method further comprising:
adding to the solution a biotinylated bioconjugate comprising a nucleotide sequence, a peptide nucleic acid sequence or a polypeptide sequence;
wherein the biotinylated bioconjugate binds to the complex.

10. The method of claim 9, wherein the biotinylated bioconjugate comprises a polynucleotide or peptide nucleic acid sequence and wherein the biological recognition event is nucleic acid hybridization of the polynucleotide sequence or peptide nucleic acid of the biotinylated bioconjugate to a target analyte.

11. The method of claim 9, wherein the biotinylated bioconjugate comprises a polypeptide sequence and a quencher, wherein the quencher is capable of amplified super-quenching of the fluorescent polymer and wherein the biological recognition event is enzyme induced cleavage of the polypeptide sequence.

12. The method of claim 10, further comprising:
adding a second bioconjugate comprising a quencher and a polynucleotide or peptide nucleic acid sequence to the solution, wherein the quencher is capable of amplified super-quenching of the fluorescent polymer and wherein the polynucleotide or peptide nucleic acid sequence of the second bioconjugate is capable of hybridizing to the polynucleotide or peptide nucleic acid sequence of the biotinylated bioconjugate.

13. The method of claim 12, wherein the polynucleotide or peptide nucleic acid sequence of the second bioconjugate is complementary to the polynucleotide or peptide nucleic acid sequence of the biotinylated bioconjugate.

14. The method of claim 11, wherein the quencher has a structure represented by the following formula: 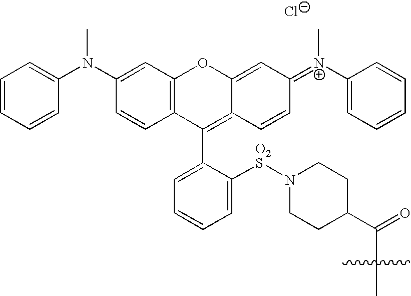
or by the formula:

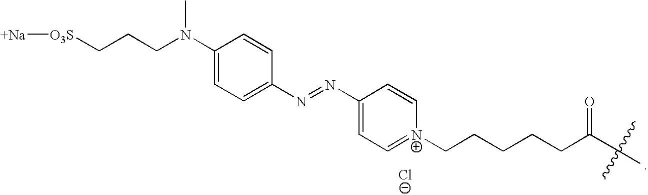

15. The method of claim 12, wherein the quencher has a structure represented by the following formula: 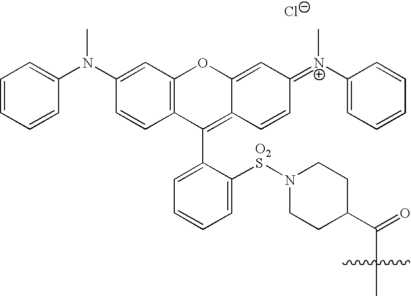
or by the formula:

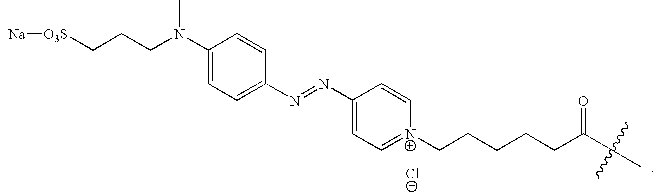

16. A sensor for detecting biological recognition events comprising:
a complex of a biotinylated fluorescent polymer and a biotin binding protein, wherein the complex comprises free biotin binding sites.

17. The sensor of claim 16, further comprising:
a solid support:
wherein the complex is disposed on a surface of the solid support.

18. The sensor of claim 16, further comprising:
a biotinylated bioconjugate comprising a polynucleotide sequence, a peptide nucleic acid sequence or a polypeptide sequence;
wherein the biotinylated bioconjugate is bound to the complex.

19. The sensor of claim 18, wherein the biotinylated bioconjugate comprises a polynucleotide sequence and wherein the biological recognition event is nucleic acid hybridization of the polynucleotide or peptide nucleic acid sequence of the biotinylated bioconjugate to a target analyte.

20. The sensor of claim 18, wherein the biotinylated bioconjugate comprises a polypeptide sequence and a quencher, wherein the quencher is capable of amplified super-quenching of the fluorescent polymer and wherein the biological recognition event is enzyme induced cleavage of the polypeptide sequence.

21. The sensor of claim 20, wherein the quencher has a structure represented by the following formula: 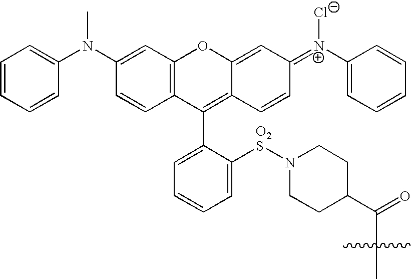
or by the formula:

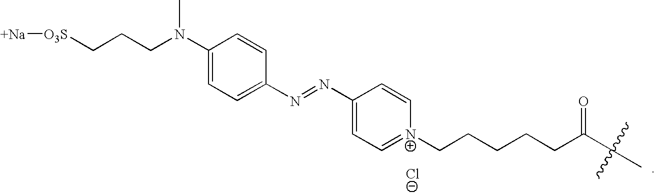

22. The sensor of claim 16, wherein the biotin binding protein is selected from the group consisting of avidin, streptavidin, and neutravidin.

23. The sensor of claim 17, wherein the solid support comprises a microsphere, a nanoparticle or a bead.

24. The sensor of claim 17, wherein the surface of the solid support comprises a functional group selected from the group consisting of ammonium functional groups, carboxylate functional groups, charged reactive groups, and neutral reactive groups.

25. The sensor of claim 17, wherein the complex further comprises:
a biotinylated bioconjugate comprising a ligand and a biotin moiety conjugated to first and second locations on a tether;
wherein the ligand comprises a quencher moiety;
wherein the quencher moiety is capable of amplified super-quenching of the fluorescent polymer;
wherein the ligand is capable of taking part in a biological recognition event; and
wherein the portion of the tether between the first and second locations has a length and a flexibility such that occurrence of the biological recognition event results in separation of the quencher from the surface of the solid support with a concomitant increase in fluorescence.

26. The sensor of claim 25, wherein the ligand comprises a polypeptide sequence.

27. The sensor of claim 25, wherein the portion of the tether between the first and second locations comprises a repeating unit represented by the chemical formula: 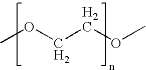
wherein n is a positive integer.

28. The sensor of claim 27, wherein n is from 70 to 80 inclusive.

29. The sensor of claim 27, wherein n is 75.

30. The sensor of claim 25, wherein the tether has a length of at least 250 Å in a fully extended conformation.

31. The sensor of claim 16, further comprising:
a biotinylated fluorescent protein;
wherein the biotinylated fluorescent protein forms a complex with the biotinylated fluorescent polymer and the biotin-binding protein.

32. The sensor of claim 31, wherein the fluorescent protein is phycoerythrin or phycobilisome.

33. A method of detecting the presence and/or amount of a target analyte in a sample comprising:
combining the sample with;
a biotinylated bioconjugate comprising a polynucleotide sequence, a peptide nucleic acid sequence or a polypeptide sequence; and
a sensor as set forth in claim 16.

34. The method of claim 33, wherein the biotinylated bioconjugate comprises a polynucleotide or peptide nucleic acid sequence, the method further comprising:
combining the sample with a second bioconjugate comprising a quencher and a polynucleotide or peptide nucleic acid sequence;
wherein the quencher is capable of amplified super-quenching of the fluorescent polymer;
wherein the polynucleotide or peptide nucleic acid sequence of the second bioconjugate is capable of hybridizing to the polynucleotide or peptide nucleic acid sequence of the biotinylated bioconjugate;
and wherein the target analyte comprises a polynucleotide sequence which is capable of hybridizing to the polynucleotide or peptide nucleic acid sequence of either the biotinylated bioconjugate or the second bioconjugate.

35. The method of claim 34, wherein the polynucleotide or peptide nucleic acid sequence of the second bioconjugate is complementary to the polynucleotide or peptide nucleic acid sequence of the biotinylated bioconjugate.

36. The method of claim 33, wherein the biotinylated bioconjugate comprises a polypeptide sequence and further comprises a quencher, wherein binding of the biotinylated bioconjugate to the complex quenches the fluorescence of the fluorescent polymer and wherein the target analyte is an enzyme capable of cleaving the polypeptide sequence.

37. A method of detecting the presence and/or amount of a target analyte in a sample comprising:
combining the sample with a sensor as set forth in claim 25.

38. The method of claim 37, wherein the ligand comprises a polypeptide sequence.

39. The method of claim 37, wherein the target analyte is selected from the group consisting of a spore, a cell, a bacteria or a virus.

40. A sensing system for detecting biological recognition events comprising:
a sensor as set forth in claim 25; and
a second solid support comprising a plurality of target moieties disposed on the surface thereof, wherein the ligand of the biotinylated bioconjugate interacts with the target moieties such that the quencher is separated from the fluorescer thereby increasing the fluorescence of the fluorescent polymer.

41. The sensing system of claim 40, wherein the ligand comprises a polypeptide sequence.

42. The sensing system of claim 40, wherein the second solid support is a microsphere, a nanoparticle or a bead.

43. A method of detecting the presence and/or amount of a target analyte in a sample comprising:
combining a sensing system as set forth in claim 40 with the sample;
wherein the target analyte can recognize and interact with the ligand and wherein interaction of the target analyte with the ligand results in a decrease in fluorescence.

44. The method of claim 43, wherein the ligand comprises a polypeptide and wherein the biological recognition event comprises the interaction of the polypeptide of the ligand with a target analyte comprising a polypeptide.

45. The method of claim 34 comprising:
incubating the sample with the biotinylated bioconjugate and the second bioconjugate; and
adding the sensor to the incubated sample.

46. The method of claim 34, wherein the sensor and the biotinylated bioconjugate are combined such that the biotinylated bioconjugate complexes to the sensor, the sample is subsequently incubated with the sensor/biotinylated bioconjugate complex, and the second bioconjugate is subsequently added to the incubated sample.

47. The method of claim 46, wherein the nucleotide sequence of the target analyte comprises a double-stranded nucleic acid, the method further comprising;
heating the incubated sample in the presence of the second bioconjugate to a temperature sufficient to melt double-stranded nucleic acid in the sample; and
cooling the sample to allow duplex formation;
wherein duplex formation between target analyte present in the sample and the second bioconjugate results in an increase in fluorescence.

48. The method of claim 46, wherein the biotinylated bioconjugate comprises a peptide nucleic acid sequence.

49. A sensor for detecting a target biological species comprising:
a bacterial spore or virus comprising a plurality of ligands for a receptor on a surface thereof;
a fluorescent polymer or fluorescent polymer complex disposed on a surface of the bacterial spore or virus; and
a plurality of bioconjugates comprising a quencher conjugated to a receptor for the ligand, wherein the receptor and ligand interact and wherein the interaction of the receptor and ligand results in amplified super-quenching of the fluorescence of the fluorescent polymer.

50. A method of detecting the presence and/or amount of a target analyte in a sample comprising:
incubating the sample with a sensor as set forth in claim 49;
wherein target analyte in the sample recognizes and interacts with the receptor and wherein interaction of target analyte in the sample and the receptor results in an increase in fluorescence.

51. The method of claim 50, wherein the target analyte comprises a bacterial spore or a virus comprising a plurality of ligands for the receptor on a surface thereof.

52. The sensor of claim 17, wherein the biotinylated bioconjugate comprises a polynucleotide or peptide nucleic acid sequence and wherein the biotinylated bioconjugate further comprises a quencher capable of amplified superquenching of the fluorescent polymer, wherein the polynucleotide sequence is located between the quencher and the biotin on the biotinylated bioconjugate.

53. A method of detecting the presence and/or amount of a target analyte in a sample comprising:
combining the sample with a sensor as set forth in claim 52;
wherein the target analyte comprises a polynucleotide sequence capable of hybridizing to the polynucleotide or peptide nucleic acid sequence of the biotinylated bioconjugate and wherein hybridization results in increased separation of the quencher from the surface of the solid support and a concomitant increase in fluorescence.

54. The method of claim 6, wherein the solid support comprises a silica or latex microsphere.

55. The sensor of claim 23, wherein the solid support comprises a silica or latex microsphere.

56. The sensing system of claim 40, wherein the surface of the second solid support comprises a functional group selected from the group consisting of ammonium functional groups, carboxylate functional groups, charged reactive groups, and neutral reactive groups.

57. The sensing system of claim 42, wherein the second solid support comprises a silica or latex microsphere.
